(19) United States
(12) Patent Application Publication
(10) Pub. No.: US 2016/0325061 A1
(43) Pub. Date: Nov. 10, 2016

(54) PATIENT VENTILATOR ASYNCHRONY DETECTION

(71) Applicant: KONINKLIJKE PHILIPS N.V., Eindhoven (NL)

(72) Inventors: CARL TAMS, EINDHOVEN (NL); NEIL EULIANO, EINDHOVEN (NL); MICHAEL BANNER, EINDHOVEN (NL); ANDREA GABRIELLI, EINDHOVEN (NL)

(21) Appl. No.: 15/110,123

(22) PCT Filed: Jan. 8, 2015
(86) PCT No.: PCT/IB2015/050143

§ 371 Date: Jul. 7, 2016

Related U.S. Application Data
(60) Provisional application No. 61/925,505, filed on Jan. 9, 2014.

(51) Int. Cl. A61M 16/00

(52) U.S. Cl. CPC A61M 16/0069; A61M 16/0051; A61M 2016/0021; A61M 2016/0027; A61M 2016/0036; A61M 2205/15; A61M 2205/3365; A61M 2205/502; A61M 2205/3368; A61M 2205/52; A61M 2230/46; A61M 2230/005

(57) ABSTRACT

Systems and methods for detecting asynchrony between a subject (106) and a ventilator (140) are based on analyzing respiratory parameters across multiple respiratory cycles. By determining the variability and/or correlation of one or more parameters related to respiratory timing (inhalation duration, exhalation duration, etc.) and/or a combination of respiratory flow rate and respiratory pressure, asynchrony may be detected and/or predicted.

### BACKGROUND

1. Field

The present disclosure pertains to systems and methods for detecting asynchrony between a subject and a ventilator during respiratory treatment of the subject using the ventilator, and, in particular, to systems and methods that determine variability and/or correlation between one or more respiratory parameters over multiple respiratory cycles.

2. Description of the Related Art

It is common to treat patients with respiratory therapy. Some examples of respiratory therapy use a (non-invasive) respiratory support circuit. Different types of respiratory support circuits may be used for different types of respiratory therapy. For some types of respiratory therapy a ventilator may be used. Some ventilators respond or react to (respiratory effort by) a patient. A failure of a ventilator to respond or react adequately to a patient, or a failure of a ventilator to provide adequate respiratory treatment, may cause asynchrony between the patient and the ventilator.

### SUMMARY

Accordingly, it is an object of one or more embodiments of the present invention to provide a system configured to non-invasively detect asynchrony between a subject and a ventilator. The system includes a ventilator, one or more sensors, and one or more processors. The ventilator is configured to provide non-invasive respiratory treatment to a subject. The one or more sensors are configured to generate output signals conveying information related to one or more respiratory parameters of the subject during multiple respiratory cycles. The one or more processors are configured to execute computer program components. The computer program components include a respiratory determination component, a statistical component, an asynchrony component, and a control component. The control component is configured to control the ventilator in accordance with a therapy regimen. The respiratory parameter component is configured to determine the one or more respiratory parameters based on the generated output signals from the one or more sensors. The statistical component is configured to determine variability across multiple respiratory cycles of the one or more determined respiratory parameters. The asynchrony component is configured to determine asynchrony between the subject and the ventilator based on the determined variability. The control component is further configured to adjust operation of the ventilator based on the asynchrony determined by the asynchrony component.

It is yet another aspect of one or more embodiments of the present invention to provide a method for non-invasively detecting asynchrony between a subject and a ventilator. The method includes generating output signals conveying information related to one or more respiratory parameters of the subject during multiple respiratory cycles; determining the one or more respiratory parameters based on the generated output signals; determining variability across multiple respiratory cycles of the one or more determined respiratory parameters; and determining asynchrony between the subject and the ventilator based on the determined variability.

It is yet another aspect of one or more embodiments to provide a system configured to non-invasively detect asynchrony between a subject and a ventilator. The system includes means for generating output signals conveying information related to one or more respiratory parameters of the subject during multiple respiratory cycles; means for determining the one or more respiratory parameters based on the generated output signals; means for determining variability across multiple respiratory cycles of the one or more determined respiratory parameters; and means for determining asynchrony between the subject and the ventilator based on the determined variability.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various FIGS. It is to be expressly understood, however, that the drawings are for the purpose of illustration and description only and are not intended as a definition of the limits of the invention.

### BRIEF DESCRIPTION OF THE DRAWINGS

FIG. 1 illustrates a schematic view of a system to detect asynchrony between a subject and a ventilator in accordance with one or more embodiments; and

FIG. 2 illustrates a method for detecting asynchrony between a subject and a ventilator in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality). As used herein, statements in parentheses may be interpreted as being optionally included, or, if the context would not permit such an interpretation, as exemplary or explanatory.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

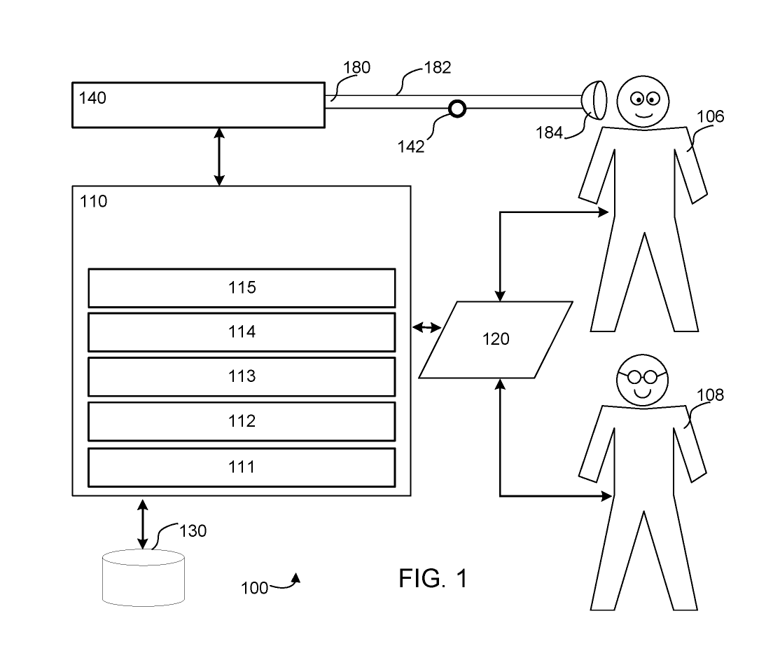

FIG. 1 illustrates a system 10 configured to (non-invasively) predict and/or detect asynchrony and/or ventilation failure between a subject 106 and a ventilator 140. Ventilator 140 is configured to provide respiratory treatment to subject 106. Ventilator may, but need not, be configured to provide non-invasive respiratory treatment. Non-invasive ventilation may be referred to as NIV. Subject 106 may interchangeably be referred to as a patient 106. System 10 may be integrated, embedded, incorporated, combined, and/or otherwise operating in conjunction with a respiratory treatment system and/or a respiratory treatment device, including but not limited to one or more of a ventilator, a positive airway pressure device (PAP/CPAP/BiPAP®/etc.), a pressure generator, and/or other systems or devices that may be used to provide respiratory treatment.

System 10 may include one or more of ventilator 140, one or more sensors 142, one or more processors 110, and/or other components. As used herein, “asynchrony” may be interpreted as the types of failures of a ventilator to adequately respond to, react to, and/or anticipate a patient's respiratory needs as may be identified by a respiratory clinician. Alternatively, and/or simultaneously, other definitions for asynchrony may be used herein.

In some embodiments, clinicians may distinguish between a set of different levels and/or scores of synchrony/asynchrony. By way of non-limiting example, a four-level set may range from severe asynchrony, to moderate asynchrony, to minor asynchrony, to synchrony. Different levels may correspond to different recommended actions, if any, to be taken on behalf of a patient. In some embodiments, an occurrence and/or detection of a particular determined level of asynchrony (e.g. “severe asynchrony”) may be referred to as a “detection of asynchrony”. In some embodiments, an occurrence and/or detection of a particular determined level of asynchrony may be referred to as a “prediction of NIV failure.” Statistically significant correlations between certain respiratory parameters (and/or parameters based thereupon) and assessments from respiratory clinicians may have been established experimentally.

Ventilator 140 may be configured to provide a pressurized flow of breathable gas for delivery to the airway of subject 106, e.g. via tubing 180. Tubing 180 may be referred to as delivery circuit 180 and/or subject interface 180. Ventilator 140 may be configured to adjust pressure levels, flow, humidity, velocity, acceleration, and/or other parameters of the pressurized flow of breathable gas in substantial synchronization with the breathing cycle of subject 106. Subject 106 may or may not initiate one or more phases of respiration. Respiratory therapy may be implemented, by way of non-limiting example, as pressure control, pressure support, volume control, flow control, and/or one or more combinations thereof. For example, to support inspiration, the pressure of the pressurized flow of breathable gas may be adjusted to an inspiratory pressure. For example, to support expiration, the pressure of the pressurized flow of breathable gas may be adjusted to an expiratory pressure. Other schemes for providing respiratory therapy through the delivery of the pressurized flow of breathable gas are contemplated within the scope of this disclosure.

A pressurized flow of breathable gas may be delivered from ventilator 140 to the airway of subject 106 via tubing 180. Tubing 180 may include a conduit 182 (e.g. a flexible length of hose) and/or a subject interface appliance 184. Tubing 180 may place subject interface appliance 184 in fluid communication with ventilator 140. Tubing 180 may form one or more flow paths through which the pressurized flow of breathable gas is communicated between subject interface appliance 184, ventilator 140, and/or system 10.

Subject interface appliance 184 may be configured to deliver the pressurized flow of breathable gas to the airway of subject 106. As such, subject interface appliance 184 may include any appliance suitable for this function. In one embodiment, ventilator 140 is a dedicated ventilation device and subject interface appliance 184 is configured to be removably coupled with another interface appliance being used to deliver respiratory therapy to subject 106. In some embodiments, the use of subject interface appliance 184 may be non-invasive. Alternatively, and/or simultaneously, in some embodiments, the use of subject interface appliance 184 may be invasive. For example, subject interface appliance 184 may be configured to engage with and/or be inserted into an endotracheal tube, a tracheotomy portal, and/or other interface appliances. In one embodiment, subject interface appliance 184 is configured to engage the airway of subject 106 without an intervening appliance. In this embodiment, subject interface appliance 184 may include one or more of an endotracheal tube, a nasal cannula, a tracheotomy tube, a nasal mask, a nasal/oral mask, a full-face mask, a total facemask, and/or other interface appliances that communicate a flow of gas with an airway of a subject. The present disclosure is not limited to these examples, and contemplates delivery of the pressurized flow of breathable gas to subject 106 using any subject interface.

Asynchrony between subject 106 and ventilator 140 may include, by way of non-limiting example, occurrences of missed triggers, extraneous triggers, delayed triggers, pre-cycling, delayed cycling, and/or other respiratory events where the respiratory needs of subject 106 are not adequately met and/or not understood. In some embodiments, asynchrony may include other occurrences, as described in this disclosure. In some embodiments, asynchrony may be caused by leaks in the respiratory support circuit (e.g. between ventilator 140 and subject 106). Asynchrony may be a predictor, among other characteristics including but not limited to rapid shallow breathing index (RSBI), pH, and partial pressure of the fraction of inspired oxygen (PaFiO₂), for a ventilation failure (or NIV failure).

In some embodiments, system 10 may include one or more sensors configured to generate output signals conveying information related to parameters of respiration, respiratory airflow, airway mechanics, physiology of subject 106, medical parameters, environmental parameters, and/or other parameters. The generated output signals may correspond to one or more respiratory cycles. FIG. 1 illustrates system 10 that includes a sensor 142 configured to generate output signals conveying information. By way of non-limiting example, parameters may include one or more of flow, (airway) pressure, humidity, velocity, acceleration, and/or other parameters. Sensor 142 may be in fluid communication with ventilator 140, system 10, and/or subject interface appliance 184. The number of sensors or the placement of sensors is not limited by the depiction in FIG. 1. The illustration of sensor 142 including one member in FIG. 1 is not intended to be limiting.

Sensor 142 is configured to generate output signals conveying information related to one or more respiratory parameters. In some embodiments, sensor 142 may be configured to generate output signals conveying information related to physiological parameters pertaining to subject 106. In some embodiments, sensor 142 may include one or more functions or features that are the same as or similar to a pressure sensor, a flow meter, a CO₂ sensor, an irradiance sensor, a light sensor, an optical sensor, a temperature sensor, a humidity sensor, a microphone, a flux sensor, and/or other sensors.

Generated output signals may convey information related to parameters associated with the state and/or condition of an airway of subject 106, the breathing of subject 106, the gas breathed by subject 106, the composition of the gas breathed by subject 106, one or more CO₂ parameters of the gas breathed by subject 106, the delivery of the gas to the airway of subject 106, and/or a respiratory effort by the subject. For example, a parameter may be related to a mechanical unit of measurement of a component of ventilator 140 (or of a device that ventilator 140 is integrated, combined, or connected with) such as valve drive current, rotor speed, motor speed, blower speed, fan speed, or a related measurement that may serve as a proxy for any of the previously listed parameters through a previously known and/or calibrated mathematical relationship. Resulting signals or information from a sensor may be transmitted to ventilator 140, processor 110, user interface 120, storage 130, and/or other components shown in FIG. 1. This transmission may be wired and/or wireless.

Output signals may be generated at a fixed and/or variable rate. For example, in some embodiments, sensor 142 may be configured to generate an output signal every 1 ms, 10 ms, 100 ms, 1 s, and/or other suitable interval.

User interface 120 of system 10 in FIG. 1 is configured to provide an interface for subject 106 (or another user 108) through which the user can provide information to and/or receive information from system 10. This enables data, results, and/or instructions and any other communicable items, collectively referred to as “information,” to be communicated between the user and system 10. An example of information that can be conveyed to subject 106 is the current mode of operation or operational setting of system 10 and/or ventilator 140. Examples of interface devices suitable for inclusion in user interface 120 include a keypad, buttons, switches, a keyboard, knobs, levers, a display screen, a touch screen, speakers, a microphone, an indicator light, an audible alarm, and a printer. Information may be provided by user interface 120 in the form of auditory signals, visual signals, tactile signals, and/or other sensory signals, or any combination thereof.

By way of non-limiting example, user interface 120 may include a radiation source capable of emitting light. The radiation source includes, for example, one or more of at least one LED, at least one light bulb, a display screen, and/or other sources. User interface 120 may control the radiation source to emit light in a manner that conveys information to subject 106.

It is to be understood that other communication techniques, either hard-wired or wireless, are also contemplated herein as user interface 120. For example, in one embodiment, user interface 120 is integrated with a removable storage interface provided by (physical) storage 130. In this example, information is loaded into system 10 from removable storage (e.g., a smart card, a flash drive, a removable disk, etc.) that enables the user(s) to customize the implementation of system 10. Other exemplary input devices and techniques adapted for use with system 10 as user interface 120 include, but are not limited to, an RS-232 port, RF link, an IR link, modem (telephone, cable, Ethernet, internet or other). In short, any technique for communicating information with system 10 is contemplated as user interface 120.

Storage 130 of system 10 in FIG. 1 comprises digital and/or electronic storage media that electronically stores information. The storage media of storage 130 may include one or both of system storage that is provided integrally (i.e., substantially non-removable) with system 10 and/or removable storage that is removably connectable to system 10 via, for example, a port (e.g., a USB port, a FireWire port, etc.) or a drive (e.g., a disk drive, etc.). Storage 130 may include one or more of optically readable storage media (e.g., optical disks, etc.), magnetically readable storage media (e.g., magnetic tape, magnetic hard drive, floppy drive, etc.), electrical charge-based storage media (e.g., EPROM, EEPROM, RAM, etc.), solid-state storage media (e.g., flash drive, etc.), and/or other electronically readable storage media. Storage 130 may store software algorithms, information determined by processor 110, information received via user interface 120, and/or other information that enables system 10 to function properly. For example, storage 130 may record or store information related to the provided respiratory therapy, and/or other information. Storage 130 may be a separate component within system 10, or is provided integrally with one or more other components of system 10 (e.g., processor 110).

Processor 110 of system 10 in FIG. 1 is configured to provide information processing and control capabilities in system 10. As such, processor 110 includes one or more of a digital processor, a microcontroller, an analog processor, a digital circuit designed to process information, an analog circuit designed to process information, a state machine, and/or other mechanisms for electronically processing information. Although processor 110 is shown in FIG. 1 as a single entity, this is for illustrative purposes only. In some implementations, processor 110 includes a plurality of processing units.

As is shown in FIG. 1, processor 110 is configured to execute one or more computer program components. The one or more computer program components include one or more of a therapy component 111, a respiratory parameter component 112, a statistical component 113, an asynchrony component 114, a control component 115, and/or other components. Processor 110 is configured to execute components 111, 112, 113, 114, and/or 115 by software; hardware; firmware; some combination of software, hardware, and/or firmware; and/or other mechanisms for configuring processing capabilities on processor 110.

It should be appreciated that although components 111-115 are illustrated in FIG. 1 as being co-located within a single processing unit, in implementations in which processor 110 includes multiple processing units, one or more of components 111-115 may be located remotely from the other components. The description of the functionality provided by the different components 111-115 described below is for illustrative purposes, and is not intended to be limiting, as any of components 111-115 may provide more or less functionality than is described. For example, one or more of components 111-115 may be eliminated, and some or all of its functionality may be provided by other ones of components 111-115. Note that processor 110 may be configured to execute one or more additional components that may perform some or all of the functionality attributed below to one of components 111-115. In some embodiments, operation of components 111-115 may be performed continuously, at variable intervals and/or at a fixed interval. For example, as new output signals are generated by sensor 142, system 10 may re-evaluate and/or re-determine the corresponding value of a parameter, variability, correlation, and/or other value that depends on the generated output signals.

Therapy component 111 is configured to obtain and/or determine a therapy regimen for subject 106. For example, a therapy regimen may be obtained from a caregiver and/or medical professional. In some embodiments, a therapy regimen may be determined based on the medical history, medical symptoms, and/or medical condition of subject 106. In some embodiments, a therapy regimen corresponds to one or more settings of system 10, and/or ventilator 140.

Respiratory parameter component 112 of system 10 in FIG. 1 is configured to determine one or more gas parameters, respiratory parameters, medical parameters, environmental parameters, and/or other parameters from output signals generated by one or more sensors 142. Determinations by respiratory parameter component 112 may be made per breathing phase, per breathing cycle, between two or more breaths, based on similarity with one or more previous breaths, and/or in other ways.

The respiratory parameters may include and/or be related to one or more of (peak) flow, flow rate, leak flow, leak correction volume, (estimated) flow limitation during exhalation, residual volume, maximum inspiratory flow per breath, (tidal) volume, (tidal) volume per minute, inhalation and/or exhalation pressure, change in pressure during the first 0.1 s of an inspiration, change in flow rate during the last 0.1 s of an exhalation, (estimated) airway resistance, (estimated) airway compliance, gas temperature, gas humidity, gas velocity, gas acceleration, gas composition (e.g. concentration(s) of one or more constituents such as, e.g., CO₂), thermal energy dissipated, (intentional) gas leak, and/or other measurements related to the (pressurized) flow of breathable gas.

One or more respiratory parameters may be derived from gas parameters and/or other output signals conveying measurements of the pressurized flow of breathable gas. The one or more respiratory parameters may include one or more of respiratory rate, breathing length or period, inhalation time or duration, exhalation time or duration, respiration flow curve shape, transition time from inhalation to exhalation and/or vice versa, transition time from peak inhalation flow rate to peak exhalation flow rate and/or vice versa, respiration pressure curve shape, maximum proximal pressure drop (per breathing cycle and/or phase), and/or other respiratory parameters, including ratios and/or other combinations of multiple respiratory parameters. For example, the respiratory parameters may include the inspiratory time of a breath divided by the breath length. Some or all of this functionality may be incorporated, shared, and/or integrated into other computer program components of processor 110.

Respiratory parameters may include timing parameters related to the respiration of subject 106, such as transitions in breathing between inhalations and exhalations. Timing parameters may include transitional moments that separate inhalation phases from exhalation phases and/or vice versa, breathing period, respiratory rate, inhalation time or duration, exhalation time or duration, start and/or end of inhalation phases, start and/or end of exhalation phases, and/or other respiratory timing parameters.

Environmental parameters may be related to one or more of the parameters of electromagnetic radiation, various temperatures, humidity level, and/or other environmental parameters, which may be related to environmental conditions near system 10 or near subject 106. One or more medical parameters may be related to monitored vital signs of subject 106, physiological parameters of subject 106, and/or other medical parameters of subject 106. Some or all of this functionality can be incorporated or integrated into other computer program components of processor 110.

Statistical component 113 is configured to determine variability, correlation, and/or similarity of one or more respiratory parameters. Statistical component 113 may be configured to support statistical operations on sets of numerical values. In some embodiments, statistical component 113 may be configured to determine one or more of breath length variability, expiratory-time variability, inspiratory-time variability, tidal volume variability, peak flow variability, leak flow variability, and/or other types of variability. In some embodiments, variability may be based on standard deviation of a number of respiratory cycles, a predetermined duration (e.g. 30, 45, 60, 90 seconds and/or another suitable duration), and/or variations and/or combinations thereof. For example, variability may be determined based on a 45-second window, and may exclude the highest and lowest values measured in that window when determining variability.

In some embodiments, statistical component 113 may be configured to determine the similarity of a current respiratory parameter (by way of non-limiting example: breath duration, inspiratory duration, expiratory duration, tidal volume, peak flow, leak flow, etc.) with one or more previously determined parameters spanning a particular number of breaths, a particular duration, and/or one or more combinations thereof.

In some embodiments, statistical component 113 may be configured to determine aggregate values (e.g. averages) of respiratory parameters spanning a particular number of breaths, a particular duration, and/or one or more combinations thereof.

In some embodiments, statistical component 113 may be configured to determine a breath profile correlation. A breath profile correlation may be based on a combination (e.g. numerical combination) of a flow correlation and a pressure correlation. For example, to determine the flow correlation, the current flow rate (during an individual respiratory cycle) may be compared to the previous 10 flow rates or an average value based thereupon (e.g. for 10 corresponding respiratory cycles). For example, to determine pressure correlation, the current inspiratory pressure may be compared to the average inspiratory pressure for the previous 10 respiratory cycles.

In some embodiments, statistical component 113 may be configured to determine breath profile consistency. Breath profile consistency may be based on a combination (e.g. numerical combination) of a flow consistency and a pressure consistency. For example, to determine the flow consistency, the current flow rate (during an individual respiratory cycle) may be compared to the previous 10 flow rates or an average value based thereupon (e.g. for 10 corresponding respiratory cycles, based on the mean absolute error between the average value and the current value).

Asynchrony component 114 is configured to determine asynchrony between subject 106 and ventilator 140. Determinations by asynchrony component 114 may be based on determinations by other computer program components and/or output signals generated by one or more sensors 142, including but not limited to one or more variabilities of respiratory parameters, breath profile correlation, breath profile consistency, and/or other measurements, determinations, and/or estimations. As used herein, the term “estimations” includes approximations.

In some embodiments, determinations by asynchrony component 114 may be based on one or more thresholds for the “r value” (and/or a value based thereon, including but not limited to r² etc.). In some embodiments, asynchrony may be expressed as a numerical value, e.g. as a synchronicity level and/or percentage.

In some embodiments, asynchrony may be modeled using an asynchrony model, which may include, by way of non-limiting example, one or more of airway resistance, expiratory time variability, leak volume, breath profile correlation, and/or other parameters.

Control component 115 is configured to control operation of system 10, system 10, and/or ventilator 140 (or components thereof), for example in accordance with a therapy regimen. Control by control component 115 may be based on determinations by other computer program components and/or output signals generated by one or more sensors 142, including, but not limited to, determinations by asynchrony component 114.

Control component 115 may be configured to control ventilator 140 such that one or more gas parameters of the pressurized flow of breathable gas are varied over time in accordance with a respiratory therapy regimen. Control component 115 may be configured to control ventilator 140 to provide the pressurized flow of breathable gas at inhalation pressure levels during inhalation phases, and at exhalation pressure levels during exhalation phases. Parameters determined by one or more components described herein, and/or received through sensors 142 may be used by control component 115, e.g. in a feedback manner, to adjust one or more therapy modes/settings/operations of system 10 and/or ventilator 140.

Alternatively, and/or simultaneously, signals and/or information received through user interface 120 may be used by control component 115, e.g. in a feedback manner, to adjust one or more therapy modes/settings/operations of system 10. Control component 115 may be configured to time its operations relative to the transitional moments in the breathing cycle of a subject, over multiple breath cycles, and/or in any other relation to any detected occurrences or determinations by timing component 112.

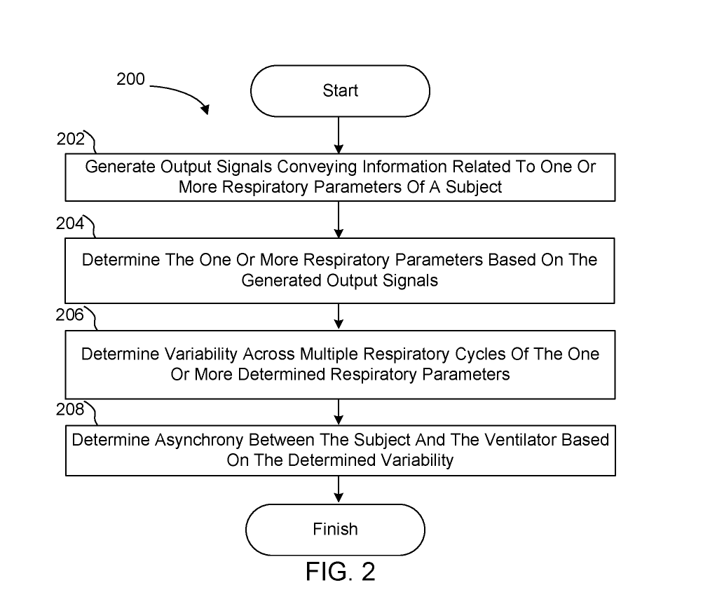

FIG. 2 illustrates a method 200 for non-invasively detecting asynchrony between a subject and a ventilator. The operations of method 200 presented below are intended to be illustrative. In some embodiments, method 200 is accomplished with one or more additional operations not described, and/or without one or more of the operations discussed. Additionally, the order in which the operations of method 200 are illustrated in FIG. 2 and described below is not intended to be limiting.

In some embodiments, method 200 is implemented in one or more processing devices (e.g., a digital processor, a microcontroller, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

At an operation 202, output signals are generated that convey information related to one or more respiratory parameters of the subject during multiple respiratory cycles. In some embodiments, operation 202 is performed by one or more sensors the same as or similar to sensor 142 (shown in FIG. 1 and described herein).

At an operation 204, the one or more respiratory parameters are determined based on the generated output signals. In some embodiments, operation 204 is performed by a respiratory parameter component the same as or similar to respiratory parameter component 112 (shown in FIG. 1 and described herein).

At an operation 206, variability is determined across multiple respiratory cycles of the one or more determined respiratory parameters. In some embodiments, operation 206 is performed by a statistical component the same as or similar to statistical component 113 (shown in FIG. 1 and described herein).

At an operation 208, asynchrony between the subject and the ventilator is determined based on the determined variability. In some embodiments, operation 208 is performed by an asynchrony component the same as or similar to asynchrony component 114 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A system configured to non-invasively detect asynchrony between a subject and a ventilator, the system comprising:
a ventilator configured to provide non-invasive respiratory treatment to a subject;
one or more sensors configured to generate output signals conveying information related to one or more respiratory parameters of the subject during multiple respiratory cycles; the one or more respiratory parameters including a flow rate and an inspiratory pressure;
one or more processors configured to execute computer program components, the computer program components comprising:
a control component configured to control the ventilator in accordance with a therapy regimen;
a respiratory parameter component configured to determine the one or more respiratory parameters based on the generated output signals from the one or more sensors such that the respiratory parameter component determines the flow rate and the inspiratory pressure based on the output signals;
a statistical component configured to determine variability across multiple respiratory cycles of the one or more respiratory parameters determined by the respiratory parameter component, the determination of variability including determining a breath profile correlation by:
comparing a current flow rate during an individual respiratory cycle to previous flow rates for previous respiratory cycles; and
comparing a current inspiratory pressure during the individual respiratory cycle to previous inspiratory pressures for previous respiratory cycles; and
an asynchrony component configured to determine asynchrony between the subject and the ventilator based on the determined variability,
wherein the control component is further configured to adjust operation of the ventilator based on the asynchrony determined by the asynchrony component.

2. The system of claim 1, wherein the one or more respiratory parameters further include one or more of breath length, inhalation duration, and/or exhalation duration.

3. The system of claim 1, wherein the one or more respiratory parameters further include one or more of tidal volume, peak flow, and/or leak flow.

4. The system of claim 1, wherein the previous flow rates for previous respiratory cycles comprise an average flow rate for multiple previous respiratory cycles and the previous inspiratory pressures for the previous respiratory cycles comprise an average inspiratory pressure for the multiple previous respiratory cycles.

5. The system of claim 1, wherein the determined asynchrony indicates a predicted failure of the provided non-invasive respiratory treatment.

6. A method for non-invasively detecting asynchrony between a subject and a ventilator, the method comprising:
generating output signals conveying information related to one or more respiratory parameters of the subject during multiple respiratory cycles; the one or more respiratory parameters including a flow rate and an inspiratory pressure;
determining the one or more respiratory parameters based on the generated output signals such that the flow rate and the inspiratory pressure are determined based on the output signals;
determining variability across multiple respiratory cycles of the one or more determined respiratory parameters, the determination of variability including determining a breath profile correlation by:
comparing a current flow rate during an individual respiratory cycle to previous flow rates for previous respiratory cycles; and
comparing a current inspiratory pressure during the individual respiratory cycle to previous inspiratory pressures for previous respiratory cycles; and
determining asynchrony between the subject and the ventilator based on the determined variability.

7. The method of claim 6, wherein the one or more respiratory parameters further include one or more of breath length, inhalation duration, and/or exhalation duration.

8. The method of claim 6, wherein the one or more respiratory parameters further include one or more of tidal volume, peak flow, and/or leak flow.

9. The method of claim 6, wherein the previous flow rates for previous respiratory cycles comprise an average flow rate for multiple previous respiratory cycles and the previous inspiratory pressures for the previous respiratory cycles comprise an average inspiratory pressure for the multiple previous respiratory cycles.

10. The method of claim 6, wherein the determined asynchrony indicates a predicted failure of the provided non-invasive respiratory treatment.

11. A system configured to non-invasively detect asynchrony between a subject and a ventilator, the system comprising:
means for generating output signals conveying information related to one or more respiratory parameters of the subject during multiple respiratory cycles, the one or more respiratory parameters including a flow rate and an inspiratory pressure;
means for determining the one or more respiratory parameters based on the generated output signals such that the flow rate and the inspiratory pressure are determined based on the output signals;
means for determining variability across multiple respiratory cycles of the one or more determined respiratory parameters, the determination of variability including determining a breath profile correlation by:
comparing a current flow rate during an individual respiratory cycle to previous flow rates for previous respiratory cycles; and
comparing a current inspiratory pressure during the individual respiratory cycle to previous inspiratory pressures for previous respiratory cycles; and
means for determining asynchrony between the subject and the ventilator based on the determined variability.

12. The system of claim 11, wherein the one or more respiratory parameters further include one or more of breath length, inhalation duration, and/or exhalation duration.

13. The system of claim 11, wherein the one or more respiratory parameters further include one or more of tidal volume, peak flow, and/or leak flow.

14. The system of claim 11, wherein the previous flow rates for previous respiratory cycles comprise an average flow rate for multiple previous respiratory cycles and the previous inspiratory pressures for the previous respiratory cycles comprise an average inspiratory pressure for the multiple previous respiratory cycles.

15. The system of claim 11, wherein the determined asynchrony indicates a predicted failure of the provided non-invasive respiratory treatment.
